(19) United States
(12) Patent Application Publication
(10) Pub. No.: US 2026/0033984 A1
(43) Pub. Date: Feb. 5, 2026

(54) SCALP COOLING DEVICE

(71) Applicant: Maria Von Med-Biotechnology Co., Ltd., New Taipei City (TW)

(72) Inventors: William Hung, New Taipei City (TW); Mei-Yue Hung Huang, New Taipei City (TW); Chun-Chou Tsai, New Taipei City (TW)

(21) Appl. No.: 18/793,818

(22) Filed: Aug. 4, 2024

(51) Int. Cl. A61F 7/10; A61F 7/00

(52) U.S. Cl. CPC A61F 7/103; A61F 2007/0002

(57) ABSTRACT

The present invention provides a scalp cooling device, including a covering part for wrapping a patient's scalp and a cooling part disposed within the covering part. This device can wrap around the patient's scalp and provide a cooling effect, causing the blood vessels in the scalp skin to constrict, reducing the blood flow to the scalp hair follicles, thereby reducing the intake of chemotherapeutic drugs by the hair follicles. Additionally, it lowers biochemical activity, thereby reducing the sensitivity of hair follicles to chemotherapy damage, and reduces the metabolic rate of the hair follicles, thus protecting the patient's hair follicles and preventing or mitigating hair loss caused by chemotherapy. Notably, the cooling part includes a gel-like coolant, which contains a moisture moderator and edible propylene glycol that maintains moisture and provides emulsifying and stabilizing effects, thus offering a long-lasting and stable cooling effect, enhancing the device's effectiveness in preventing hair loss.

### FIELD OF THE INVENTION

The present disclosure belongs to the technical field of medical auxiliary devices, specifically relating to a scalp cooling device.

### BACKGROUND OF THE INVENTION

Currently, cancer treatment methods mainly include surgical removal of lesions, chemotherapy, radiation therapy, targeted drug therapy, and some other treatment methods. Among them, chemotherapy is a very commonly used treatment method. Common adverse reactions caused by chemotherapy include severe hair loss due to the cytotoxicity of chemotherapeutic drugs, causing psychological harm and burden to patients. Many patients, therefore, opt for additional anti-hair loss treatment. In anti-hair loss treatments, the patient's scalp is usually cooled. Research has shown that cooling can cause the blood vessels in the scalp skin to constrict, reducing the blood flow to the scalp hair follicles, thus reducing the intake of chemotherapeutic drugs by the hair follicles. Cooling also lowers biochemical activity, thereby reducing the sensitivity of hair follicles to chemotherapy damage, and reduces the metabolic rate of the hair follicles, thus protecting the patient's hair follicles and preventing or mitigating hair loss caused by chemotherapy.

There are some existing scalp cooling devices used for anti-hair loss treatment, which can be mainly divided into two categories. One category includes a cap body and a cooling system that continuously supplies a coolant to the cap body. These devices are large in size, can only be used in specific environments, are inconvenient to use, and are expensive. The other category is a portable cooling cap, which includes a cap body and a coolant provided on the cap body. This type of device needs to be cooled before use and then worn on the patient's head, making it more convenient to use and less expensive, but it often fails to achieve good cooling effects. For example, cooling caps using ice packs have very low temperatures when first worn, resulting in poor patient experience; as the ice in the ice pack melts, the cooling effect quickly diminishes and becomes uneven, leading to poor anti-hair loss treatment results.

### SUMMARY OF THE INVENTION

The present disclosure aims to solve the above problems by providing a scalp cooling device that can better prevent or mitigate hair loss caused by chemotherapy and is convenient to use. The invention adopts the following technical solutions.

The present disclosure provides a scalp cooling device for cooling the scalp of chemotherapy patients, including: a covering part for wrapping the scalp; and one or more cooling parts disposed within the covering part for cooling the scalp, wherein the cooling part includes a gel-like coolant comprising a moisture moderator, water, methylcellulose, and edible propylene glycol.

In some embodiments, the covering part has multiple strip-shaped accommodating parts, the cooling part comprises multiple cooling parts respectively filled in each accommodating part.

In some embodiments, the distribution of the multiple cooling parts and the capacity of the coolant in each cooling part ensure that after the chemotherapy patient wears the scalp cooling device for a first predetermined time, and, during a second predetermined time while continuing to wear the scalp cooling device, the temperature of the scalp remains about 12° C. to 28° C., 14° C. to 28° C., 16° C. to 28° C., 18° C. to 28° C., 20° C. to 28° C., 22° C. to 28° C., 24° C. to 28° C., 26° C. to 28° C., 12° C. to 26° C., 12° C. to 24° C., 12° C. to 22° C., 12° C. to 20° C., 12° C. to 18° C., 12° C. to 16° C., or 12° C. to 14° C. In some embodiments, during a second predetermined time while continuing to wear the scalp cooling device, the preferable temperature of the scalp remains about 12° C., 14° C., 16° C., 18° C., 20° C., 22° C., 24° C., 26° C. or 28° C.

In some embodiments, the covering part of the scalp cooling device in the present disclosure includes: a top covering piece in a curved sheet form with arc-shaped side edges, and two side covering pieces respectively provided on both sides of the top covering piece. Further, one end of the side covering piece in the length direction being semi-circular and connected to the corresponding side edge.

In some embodiments, the top covering piece of the scalp cooling device in the present disclosure has multiple first accommodating parts as the accommodating parts, the multiple first accommodating parts are sequentially arranged along the curved direction of the top covering piece, and the long sides of two adjacent first accommodating parts face each other; the side covering piece has multiple second accommodating parts as the accommodating parts, the multiple second accommodating parts are sequentially arranged along the length direction of the side covering piece, and the long sides of two adjacent second accommodating parts face each other.

In some embodiments, the width of the multiple first accommodating parts located in the middle of the top covering piece is the same. In some embodiments, preferably, the width of the multiple first accommodating parts located in the middle of the top covering piece is about 3 to 6 cm, 3.5 to 6 cm, 4 to 6 cm, 4.5 to 6 cm, 5 to 6 cm, 5.5 to 6 cm, 3 to 5.5 cm, 3 to 5 cm, 3 to 4.5 cm, 3 to 4 cm, or 3 to 3.5 cm. In some embodiments, much preferably, the width of the multiple first accommodating parts located in the middle of the top covering piece is about 3 cm, 3.5 cm, 4 cm, 4.5 cm, 5 cm, 5.5 cm, or 6 cm.

In some embodiments, the width of the multiple second accommodating parts is the same. In some embodiments, the width of the multiple second accommodating parts preferably is about 4 to 7.5 cm, 4 to 7 cm, 4 to 6.5 cm, 4 to 6 cm, 4 to 5.5 cm, 4 to 5 cm, 4 to 4.5 cm, 4.5 to 7.5 cm, 5 to 7.5 cm, 5.5 to 7.5 cm, 6 to 7.5 cm, 6.5 to 7.5 cm, or 7 to 7.5 cm. In some embodiments, the width of the multiple second accommodating parts much preferably is about 4 cm, 4.5 cm, 5 cm, 5.5 cm, 6 cm, 6.5 cm, 7 cm, or 7.5 cm.

In some embodiments, the top covering piece has at least 8 of the first accommodating parts, and each side covering piece has at least 3 of the second accommodating parts.

In some embodiments, the top covering piece includes a first outer layer and a first inner layer stacked together, and multiple first partition parts. The first partition parts divide the space between the stacked first outer layer and first inner layer into multiple first accommodating parts. The side covering piece includes a second outer layer and a second inner layer stacked together, and multiple second partition parts, the second partition parts divide the space between the stacked second outer layer and second inner layer into multiple second accommodating parts.

In some embodiments, the top covering piece further includes a first edge-wrapping piece wrapping the edge of the stacked first outer layer and first inner layer, and the side covering piece further includes a second edge-wrapping piece wrapping the edge of the stacked second outer layer and second inner layer.

In some embodiments, the materials of the first outer layer, second outer layer, first inner layer, and second inner layer are all polyester fiber and cotton blended fabric. The material of the first edge-wrapping piece is nylon. The materials of the first partition part and second partition part are polyethylene plastic.

Compared to the prior art, the present application has at least the following beneficial effects or unexpected results:

According to the present disclosure, the present scalp cooling device includes a covering part for wrapping the patient's scalp and a cooling part provided in the covering part. This design allows the device to wrap around the patient's scalp and provide a cooling effect. The cooling effect causes the blood vessels in the scalp skin to constrict, reducing the blood flow to the hair follicles in the scalp, thereby decreasing the uptake of chemotherapeutic drugs by the hair follicles. Additionally, the cooling reduces biochemical activity, lowering the sensitivity of hair follicles to chemotherapy damage and reducing the metabolic rate of the hair follicles, thus protecting the patient's hair follicles and preventing or reducing hair loss caused by chemotherapy.

Notably, the present cooling part includes a gel-like coolant, which contains a moisture-retaining agent and edible propylene glycol that maintains moisture and provides emulsifying and stabilizing effects. This composition ensures a long-lasting and stable cooling effect, enhancing the device's effectiveness in preventing hair loss.

### BRIEF DESCRIPTION OF DRAWINGS

One or more specific embodiments are shown in the accompanying drawings by way of example and not by way of limitation. The accompanying drawings are not to scale unless otherwise disclosed. The present disclosure should be understood by those of ordinary knowledge in the art in light of the following detailed description of preferred specific embodiments and with reference to the accompanying drawings.

The above features, technical characteristics, advantages, and their realizations of the present disclosure will be further described below in a clear and easy-to-understand manner in conjunction with the illustrations of the preferred specific embodiments.

FIG. 1 is a perspective view of the scalp cooling device in the embodiment of the present invention.

FIG. 2 is a front view of the scalp cooling device in the embodiment of the present invention.

FIG. 3 is a side view of the scalp cooling device in the embodiment of the present invention.

FIG. 4 is a schematic structural view of the top covering piece of the scalp cooling device in the embodiment of the present invention in a flat state.

FIG. 5 is a schematic structural view of the side covering piece of the scalp cooling device in the embodiment of the present invention in a flat state.

FIG. 6 is a comparison chart of scalp temperatures before and after using the scalp cooling device in the embodiment of the present invention.

### DETAILED DESCRIPTION OF THE INVENTION

To make the technical means, creation features, achieved purposes, and effects of the present invention easy to understand, the following specific embodiments and accompanying drawings illustrate the scalp cooling device of the present invention.

### Embodiment

This embodiment provides a scalp cooling device for cooling the scalp of cancer patients who need chemotherapy, thus preventing or mitigating hair loss caused by chemotherapy.

Our relevant research shows that in the human keratinocyte model, lowering the temperature to 22° C. or below, especially further lowering it to 18° C. or even 14° C., can better avoid the cytotoxicity of chemotherapeutic drugs. Therefore, the scalp cooling device of this embodiment aims to maintain the patient's scalp temperature within the range of 14° C. to 22° C. during the predetermined anti-hair loss treatment period.

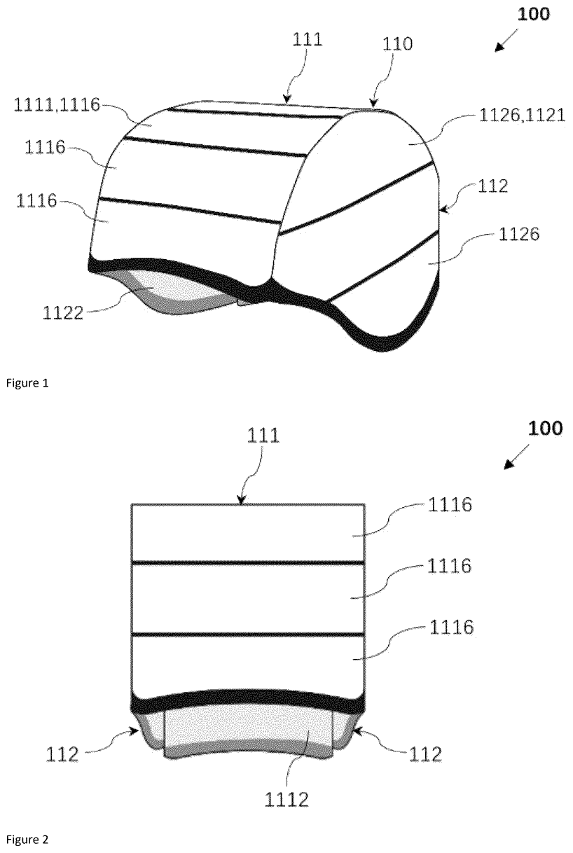

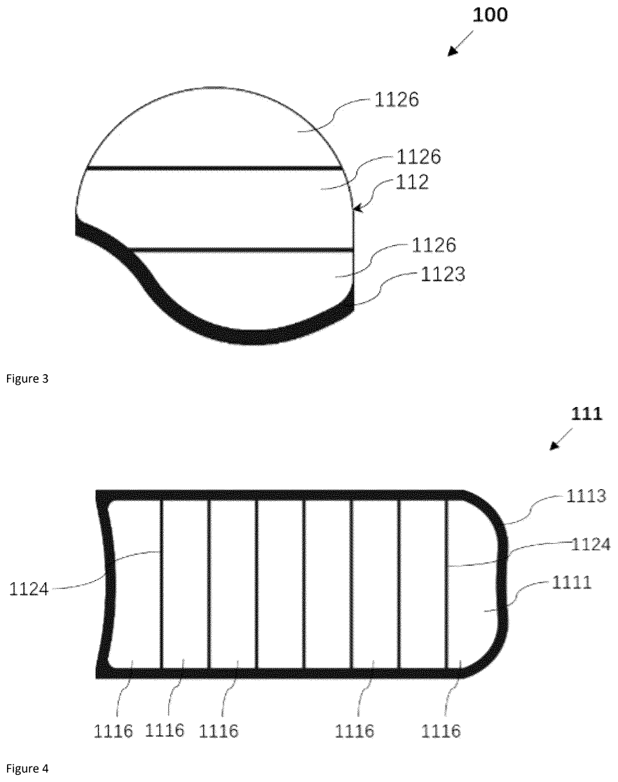

FIG. 1 is a perspective view of the scalp cooling device in this embodiment. FIG. 2 is a front view of the scalp cooling device in this embodiment. FIG. 3 is a side view of the scalp cooling device in this embodiment.

As shown in FIGS. 1 to 3, the scalp cooling device 100 of this embodiment includes a covering part 110, an anti-slip fixing part, and multiple cooling parts (not shown in the figure) provided within the interlayer of the covering part 110.

The covering part 110 is generally hat-shaped and can substantially cover the top of the patient's head, the back of the head, and large areas on both sides of the head when worn on the patient's head. The covering part 110 includes a top covering piece 111 and two side covering pieces 112.

FIG. 4 is a schematic structural view of the top covering piece in a flat state in this embodiment.

As shown in FIGS. 1 to 3, in the scalp cooling device 100 as a product, the top covering piece 111 is in a curved sheet form; as shown in FIG. 4, in a flat state, the top covering piece 111 is generally in a rectangular sheet form with slightly curved opposite sides, with the two sides in the width direction being generally straight, one side in the length direction being convexly curved, and the other side in the length direction being concavely curved.

The top covering piece 111 includes a first outer layer 1111, a first inner layer 1112, a first edge-wrapping piece 1113, multiple first partition parts 1114, and a first waterproof layer (not shown in the figure).

In the flat state, the outer contour shapes of the first outer layer 1111 and the first inner layer 1112 are similar to the outer contour shape of the top covering piece 111 in the flat state, and the first outer layer 1111 and the first inner layer 1112 are stacked to form a double-layer structure. The first edge-wrapping piece 1113 wraps the entire edge of the stacked first outer layer 1111 and first inner layer 1112. The first waterproof layer is provided inside the stacked first outer layer 1111 and first inner layer 1112, preventing the internal coolant from leaking out. The first partition parts 1114 are strip-shaped and provided at corresponding positions on the stacked first outer layer 1111 and first inner layer 1112, dividing the space between the first outer layer 1111 and first inner layer 1112 into multiple strip-shaped first accommodating parts 1116 for accommodating the cooling parts. The length direction of the first accommodating parts 1116 is the width direction of the top covering piece 111 in the flat state, and multiple first accommodating parts 1116 are sequentially arranged along the length direction of the top covering piece 111 in the flat state.

In this embodiment, the materials of the first outer layer 1111 and first inner layer 1112 are polyester fiber and cotton blended fabric. The material of the first edge-wrapping piece 1113 is nylon. The first partition parts 1114 are adhesive parts formed from polyethylene plastic.

In the flat state, the overall length of the top covering piece 111 is 39.2 cm, and the overall width is 18 cm. The width of the first edge-wrapping piece 1113 is 1 cm. The width of the first partition parts 1114 is 0.3 cm. The top covering piece 111 has eight first accommodating parts 1116, wherein the width of one first accommodating part 1116 located at the convex edge is 4.6 cm, the width of the remaining seven first accommodating parts 1116 is 4.2 cm, and the width of one first accommodating part 1116 located at the concave edge is 4.9 cm.

FIG. 5 is a schematic structural view of the side covering piece in a flat state in this embodiment.

As shown in FIGS. 1 to 3, in the scalp cooling device 100 as a product, the side covering piece 112 is in a slightly convex sheet form; as shown in FIG. 5, in a flat state, the side covering piece 112 is in a sheet form with curved edges, with one side in the width direction being semi-circular and the other side in the width direction being close to an S-shape. The semi-circular side of the side covering piece 112 is connected to the curved side edges of the top covering piece 111 in a curved state, forming an overall hat shape.

The side covering piece 112 includes a second outer layer 1121, a second inner layer 1122, a second edge-wrapping piece 1123, second partition parts 1124, and a second waterproof layer (not shown in the figure).

Similarly, the outer contour shapes of the second outer layer 1121 and the second inner layer 1122 in a flat state are similar to the outer contour shape of the side covering piece 112 in a flat state, and the two are stacked to form a double-layer structure. The second edge-wrapping piece 1123 wraps the entire edge of the stacked second outer layer 1121 and second inner layer 1122. The second waterproof layer is provided inside the stacked second outer layer 1121 and second inner layer 1122, preventing the internal coolant from leaking out. The second partition parts 1124 are strip-shaped and provided at corresponding positions on the stacked second outer layer 1121 and second inner layer 1122, dividing the space between the second outer layer 1121 and second inner layer 1122 into multiple strip-shaped second accommodating parts 1126 for accommodating the cooling parts. The length direction of the second accommodating parts 1126 is the length direction of the side covering piece 112 in the flat state, and multiple second accommodating parts 1126 are sequentially arranged along the width direction of the side covering piece 112 in the flat state.

In this embodiment, the materials of the second outer layer 1121 and second inner layer 1122 are also polyester fiber and cotton blended fabric. The material of the second edge-wrapping piece 1113 is also nylon. The second partition parts 1114 are also adhesive parts formed from polyethylene plastic.

The overall length of the side covering piece 112 is 21.2 cm, and the overall width is 18.6 cm. The width of the second edge-wrapping piece 1123 is 1 cm. The width of the second partition parts 1124 is 0.3 cm. The side covering piece 112 has three second accommodating parts 1126, each with a width of 5.3 cm.

The anti-slip fixing part is an elastic strap, with both ends fixed to the outer ends of the two side covering pieces 112, for securing the scalp cooling device 100 under the patient's chin when worn, preventing accidental detachment during the anti-hair loss treatment process.

The cooling part includes a gel-like coolant comprising a moisture moderator, water, methylcellulose, and edible propylene glycol. Optionally, the cooling part also includes a water-blocking isolation layer wrapped around the gel-like coolant. Multiple cooling parts are respectively filled in the accommodating parts of the top covering piece 111 and side covering piece 112, i.e., each accommodating part contains a gel-like coolant corresponding to its shape, and the coolant is evenly distributed throughout the covering part 110. When the scalp cooling device 100 is worn on the patient's head, the length direction of the multiple accommodating parts is generally perpendicular to the direction of gravity, effectively preventing or limiting the movement (displacement) of the coolant due to gravity, keeping the coolant evenly distributed throughout the covering part 110.

The distribution of the multiple cooling parts and the composition and capacity of the coolant in each cooling part ensure that after the patient wears the scalp cooling device 100 for a first predetermined time, the temperature of the scalp can be maintained within the predetermined temperature range during a second predetermined time while continuing to wear the scalp cooling device 100.

In this embodiment, the distribution of the multiple cooling parts and the composition and capacity of the coolant in each cooling part ensure that after the patient wears the scalp cooling device 100 for 15 minutes, the scalp temperature can reach 14° C. to 22° C., and after continuing to wear the scalp cooling device 100 for at least 120 minutes, the scalp temperature can be maintained within this temperature range.

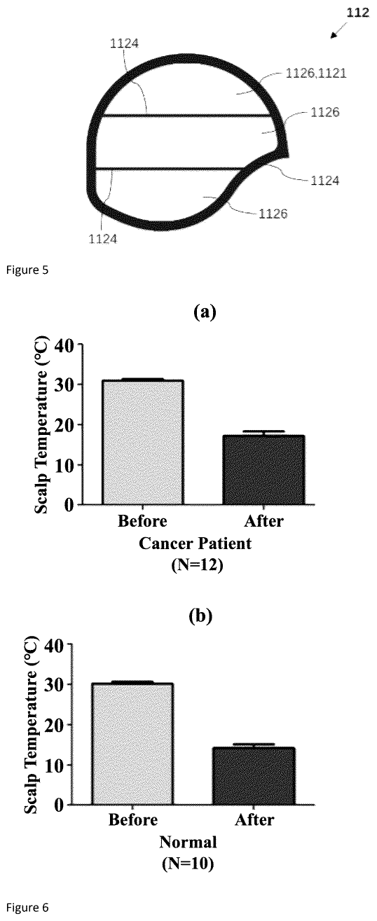

FIG. 6 is a comparison chart of scalp temperatures before and after using the scalp cooling device in this embodiment, wherein (a) is a bar chart of scalp temperatures of cancer patients before and after wearing the scalp cooling device, and (b) is a bar chart of scalp temperatures of normal people (i.e., non-cancer patients) before and after wearing the scalp cooling device.

As shown in FIG. 6, before wearing, the scalp temperature of multiple cancer patients was slightly over 30° C.; after wearing the scalp cooling device 100 for 15 minutes, the lowest scalp temperature of cancer patients dropped to 13.73° C., with the average temperature of multiple patients reaching 17.16° C. after 15 minutes of wearing.

Before wearing, the scalp temperature of multiple normal people was about 30° C.; after wearing the scalp cooling device 100 for 15 minutes, the lowest scalp temperature of normal people dropped to 15.93° C., with the average temperature of multiple people reaching 14.14° C. after 15 minutes of wearing.

This embodiment also provides a method of using the above scalp cooling device, including the following steps:
Step S1 (preparation step), cooling the scalp cooling device 100 using refrigeration equipment to bring the cooling parts to a low-temperature state.

Optionally, the refrigeration equipment is a household refrigerator, with the temperature of the refrigerator compartment generally around 4° C., and the temperature of the freezer compartment generally −15° C. or lower. In step S1, the scalp cooling device 100 can be placed in a storage bag, which is then placed in the freezer compartment of a household refrigerator for cooling for 6 hours or more. After that, the storage bag and the scalp cooling device 100 are taken out, the scalp cooling device 100 is removed from the storage bag, and the moisture on the surface of the scalp cooling device 100 caused by the temperature difference is wiped off, then it can be worn and used.
Step S2 (cooling step), during the chemotherapy period (during the administration of the drug), the first predetermined period before the chemotherapy period, or the second predetermined period after the chemotherapy period, cooling the patient's scalp by letting the patient wear the cooled scalp cooling device 100, thereby preventing or mitigating hair loss caused by chemotherapy.

Preferably, in step S2, letting the patient wear the cooled scalp cooling device 100 for 30 minutes before chemotherapy and 90 minutes during chemotherapy.

Optionally, in step S2, the temperature of the patient's scalp is monitored using a temperature monitoring system to ensure the scalp temperature remains within the desired range. The temperature monitoring system may include a temperature sensor placed between the patient's scalp and the scalp cooling device 100, and a receiving and processing device that receives the signal from the temperature sensor, converts it into the corresponding temperature value, and displays it. When the scalp temperature exceeds the desired temperature range, the patient can be promptly allowed to replace it with another sufficiently cooled scalp cooling device 100, thereby ensuring the effectiveness of the anti-hair loss treatment.

Additionally, normal people can also use the scalp cooling device 100 of this embodiment, for example, for head cooling during a fever, for cold compresses when the head is injured, or to prevent hair loss caused by follicle blockage due to excessive sweating in hot weather.

When used by normal people, the scalp cooling device 100 can be placed in a storage bag, which is then placed in the freezer or refrigerator compartment of a household refrigerator for cooling for 6 hours or more. After that, the storage bag and the scalp cooling device 100 are taken out, the scalp cooling device 100 is removed from the storage bag, the moisture on the surface of the scalp cooling device 100 is wiped off, and then it can be worn and used.

### Effect and Benefit of the Embodiment

According to the scalp cooling device provided by this embodiment, the covering part wraps the patient's scalp, and the cooling part is disposed within the covering part, thereby wrapping the patient's scalp and providing a cooling effect, causing the blood vessels in the scalp skin to constrict, reducing the blood flow to the scalp hair follicles, thereby reducing the intake of chemotherapeutic drugs by the hair follicles. It also lowers biochemical activity, reducing the sensitivity of hair follicles to chemotherapy damage, and reduces the metabolic rate of the hair follicles, thus protecting the patient's hair follicles and preventing or mitigating hair loss caused by chemotherapy. Notably, the cooling part includes a gel-like coolant that contains a moisture moderator and edible propylene glycol capable of retaining moisture and providing emulsifying and stabilizing effects, thus offering a long-lasting and stable cooling effect, enhancing the device's effectiveness in preventing hair loss.

Furthermore, the covering part has multiple strip-shaped accommodating parts, each filled with gel-like coolant, ensuring that the coolant is evenly distributed throughout the covering part. This allows the patient's scalp temperature to quickly reach the desired range after wearing and maintain it throughout the anti-hair loss treatment, providing stable and long-lasting cooling effects, thereby improving the anti-hair loss treatment effect.

Additionally, the scalp cooling device can maintain the scalp temperature within 14° C. to 22° C. throughout the anti-hair loss treatment, better preventing the cytotoxic effects of the drug on the hair follicles, thus improving the anti-hair loss treatment effect.

Moreover, the covering part is formed by combining a top covering piece and two side covering pieces, with multiple first accommodating parts arranged along the curved direction of the top covering piece and multiple second accommodating parts arranged along the length direction of the side covering pieces. This structure effectively prevents or limits the movement of the coolant, ensuring that it remains evenly distributed throughout the covering part, providing a more uniform cooling effect. Additionally, this covering part structure is easier to manufacture.

Furthermore, the surface materials of the top covering piece and side covering pieces are polyester fiber and cotton blended fabric, and the edge-wrapping materials are nylon, making the structure strong and reliable, wear-resistant, dimensionally stable under temperature changes, and easy to maintain and clean, thus being more convenient to use.

The above embodiment is only used to illustrate the specific implementation of the invention. The invention is not limited to the described range of the embodiment. A person skilled in the art should understand that the invention is not limited by the above embodiment. The principles of the invention are described in the above embodiment and the specification. There may be various changes and improvements within the spirit and scope of the invention, and these changes and improvements fall within the scope of the invention as claimed.

1. A scalp cooling device for cooling a scalp of a chemotherapy patient, comprising:
a covering portion for covering the scalp; and
a cooling portion provided in the covering portion for cooling the scalp,
wherein the cooling portion comprises a gel-like coolant, the coolant comprising: a moisture-retaining agent, water, methylcellulose, and edible propylene glycol.

2. The scalp cooling device according to claim 1, wherein the covering portion has a plurality of strip-shaped accommodating parts, and the cooling portion comprises a plurality of cooling portions respectively filled in each of the accommodating parts.

3. The scalp cooling device according to claim 2, wherein a distribution of the plurality of cooling portions and a capacity of the coolant in each of the cooling portions ensure that after the chemotherapy patient wears the scalp cooling device for a first predetermined time, a temperature of the scalp remains between about 12° C. to 28° C. during a second predetermined time while continuing to wear the scalp cooling device.

4. The scalp cooling device according to claim 2, wherein the covering portion comprises:
a top covering piece in a curved sheet form with an arc-shaped side edge; and
two side covering pieces respectively provided on both sides of the top covering piece, one end of the side covering piece in the length direction being semi-circular and connected to the corresponding side edge.

5. The scalp cooling device according to claim 4, wherein the top covering piece comprises: a plurality of first accommodating parts as the accommodating parts, the plurality of first accommodating parts are sequentially arranged along the curved direction of the top covering piece, and the long sides of two adjacent first accommodating parts face each other; and
the side covering piece has a plurality of second accommodating parts as the accommodating parts, the plurality of second accommodating parts are sequentially arranged along the length direction of the side covering piece, and the long sides of two adjacent second accommodating parts face each other.

6. The scalp cooling device according to claim 5, wherein a width of the plurality of first accommodating parts located in the middle of the top covering piece is the same.

7. The scalp cooling device according to claim 6, wherein the width is about 3 to 6 cm.

8. The scalp cooling device according to claim 5, wherein a width of the plurality of second accommodating parts is the same.

9. The scalp cooling device according to claim 8, wherein the width is about 4 to 7.5 cm.

10. The scalp cooling device according to claim 5, wherein the top covering piece has at least 8 of the first accommodating parts, and each of the side covering pieces has at least 3 of the second accommodating parts.

11. The scalp cooling device according to claim 4, wherein the top covering piece further comprises a first outer layer and a first inner layer stacked together, and a plurality of first partition parts, the plurality of first partition parts dividing the space between the stacked first outer layer and the first inner layer into the plurality of first accommodating parts; the side covering piece comprises a second outer layer and a second inner layer stacked together, and a plurality of second partition parts, the plurality of second partition parts dividing the space between the stacked second outer layer and the second inner layer into the plurality of second accommodating parts.

12. The scalp cooling device according to claim 4, wherein the top covering piece further comprises a first edge-wrapping piece wrapping the edge of the stacked first outer layer and the first inner layer, and the side covering piece further comprises a second edge-wrapping piece wrapping the edge of the stacked second outer layer and the second inner layer.

13. The scalp cooling device according to claim 11, wherein the materials of the first outer layer, the second outer layer, the first inner layer, and the second inner layer are all polyester fiber and cotton blended fabric, the material of the first edge-wrapping piece is nylon, and the materials of the first partition part and the second partition part are polyethylene plastic.
